(19) United States
(12) Patent Application Publication
(10) Pub. No.: US 2020/0352614 A1
(43) Pub. Date: Nov. 12, 2020

(54) Apparatus with an Anti-Backout Feature and Related Method

(71) Applicant: FBC Device ApS, Risskov (DK)

(72) Inventors: Michael Orabona, Lowell, MA (US); Andrew Galanis, Ipswich, MA (US); Finn Bjarke Christensen, Silkeborg (DK); Bruce H. Robie, North Andover, MA (US)

(73) Assignee: FBC Device ApS, Risskov (DK)

(21) Appl. No.: 16/867,588

(22) Filed: May 6, 2020

Related U.S. Application Data
(60) Provisional application No. 62/843,760, filed on May 6, 2019.

(51) Int. Cl. A61B 17/80

(52) U.S. Cl. CPC A61B 17/8052

(57) ABSTRACT

Embodiments of the present disclosure are drawn to an apparatus, and related method, with an anti-backout feature. An exemplary apparatus described herein includes a block or plate formed with a cavity that is configured receive a device, wherein the block comprises at least two holes, and wherein each hole further comprises a first portion and a second portion; and an elongate lockout feature comprising first and second ends, wherein the first and second ends further comprise a cross-sectional profile, and wherein the first and second ends of the elongate lockout feature are seated within the respective at least two holes in the block to prevent the device from backing out of the cavity of the block.

### CROSS-REFERENCE TO RELATED APPLICATION

The present application claims priority benefit to a provisional application entitled “Apparatus with an Anti-Backout Feature and Related Method,” which was filed on May 6, 2019, and assigned Ser. No. 62/843,760. The entire content of the foregoing provisional application is incorporated herein by reference.

### TECHNICAL FIELD

The present disclosure relates to an apparatus, and related method, with an anti-backout feature. In some instances, embodiments of the present disclosure relate to an apparatus with an anti-backout feature that may be used for a variety of orthopedic uses, such as, e.g., plate(s) using one or more screws to affix the plate(s) to one or more bones in the body or block(s) using one or more screws (or other suitable device) to affix the block(s) to one or more bones in the body. In some uses, the plate(s) or block(s) may be affixed to other plates(s) or block(s), which are then implanted into the body.

### SUMMARY

Aspects of the present disclosure relate to one or more devices having a geometry for an anti-backout feature. According to aspects of the present disclosure and exemplary embodiments described herein, an anti-backout feature (e.g., a locking wire or a locking feature) may be placed, configured and/or positioned within a non-linear hole to prevent a finally assembled object or device from backing out or being ejected from a cavity of a block (e.g., a block or a plate used during orthopedic applications). In an exemplary embodiment, a non-linear hole may be disposed within a block or a plate to ensure that the anti-backout feature (e.g., locking wire or locking feature) will not fall out from the block or the plate. Aspects of the present disclosure may take advantage of 3D printing to create the opening and holes, which may previously have been difficult to manufacture.

In an exemplary embodiment of the present disclosure, the apparatus may include a block or plate formed with a cavity that is configured to receive a device, wherein the block includes at least two holes, and wherein each hole further includes a first portion and a second portion; and an elongate lockout feature that includes first and second ends, wherein the first and second ends define a cross-sectional profile, and wherein the first and second ends of the elongate lockout feature are seated within the respective at least two holes in the block to prevent the device from backing out of the cavity of the block.

In a further exemplary embodiment of the present disclosure, the disclosed method may include inserting a device into a block or plate formed with a cavity configured receive to device, wherein the block is formed with at least two holes, and wherein each hole includes a first portion and a second portion; and securing the device within the block or plate by inserting an elongate lockout feature into the cavity of the block or plate, wherein the elongate lockout feature includes first and second ends, wherein the first and second ends define a cross-sectional profile, and wherein the first and second ends of the elongate lockout feature are seated within the respective at least two holes in the block to prevent the device from backing out of the cavity of the block.

It may be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate exemplary embodiments of the present disclosure and together with the description, serve to explain the principles of the disclosure.

FIG. 1 is a perspective view of an exemplary embodiment depicted as a block, according to an aspect of the present disclosure;

FIG. 2 is a front view of the exemplary block of FIG. 1;

FIG. 3 is a side elevation view of the exemplary block of FIG. 1;

FIG. 4 is a cross-sectional view of the exemplary block of FIG. 1, along line A-A in FIG. 3;

FIG. 5 is a cross-sectional view of the exemplary block of FIG. 1, along line A-A in FIG. 3;

FIG. 6 is a cross-sectional side elevation view aligned with a middle of a cavity of the exemplary block of FIG. 1;

FIG. 7 is a perspective view of an alternative exemplary embodiment depicted as a block, according to an aspect of the present disclosure;

FIG. 8 is a front view of the exemplary block of FIG. 7;

FIG. 9 is a side elevation view of the exemplary block of FIG. 7;

FIG. 10 is a cross-sectional view of the exemplary block of FIG. 7, along line C-C in FIG. 9;

FIG. 11 is a cross-sectional side elevation view aligned with a middle of a cavity of the exemplary block of FIG. 7;

FIG. 12 is a front view of an alternative exemplary embodiment depicted as a block, according to an aspect of the present disclosure;

FIG. 13 is a side elevation view of the exemplary block of FIG. 12;

FIG. 14 is a cross-sectional view of the exemplary block of FIG. 12, along line C-C in FIG. 13; and

FIG. 15 is a cross-sectional side elevation view of the exemplary block of FIG. 12, along line B-B in FIG. 12;

FIG. 16 is an isometric view of an alternative embodiment depicted as a block, according to an aspect of the present disclosure;

FIG. 17 is a front view of the exemplary block of FIG. 16;

FIG. 18 is a side elevation view of the exemplary block of FIG. 16;

FIG. 19 is a cross-sectional view of the exemplary block of FIG. 16, along line C-C in FIG. 18, with a locking feature in its undeformed shape; and

FIG. 20 is a cross-sectional view of the exemplary block of FIG. 16, along line B-B in FIG. 17, with a locking feature in its undeformed shape; and

FIG. 21 is an alternate embodiment of the exemplary block of FIG. 16, along line C-C in FIG. 18, showing a different hole configuration; and

FIG. 22 is a front view (aligned with the orientation of FIG. 14 and FIG. 19) of an alternate embodiment of a locking feature; and

FIG. 23 is a front view (aligned with the orientation of FIG. 14 and FIG. 19) of an alternate embodiment of a locking feature.

### DETAILED DESCRIPTION OF CERTAIN EXEMPLARY EMBODIMENTS

Reference will now be made in detail to certain exemplary embodiments according to the present disclosure, certain examples of which are illustrated in the accompanying drawings. Wherever possible, the same reference numbers will be used throughout the drawings to refer to the same or like parts.

In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Any range described herein will be understood to include the endpoints and all values between the endpoints.

The section headings used herein are for organizational purposes only and are not to be construed as limiting the subject matter described.

An advantage of the geometries for an anti-backout feature, as taught herein, is to prevent back out (or ejection) of an assembled device from a cavity formed within or by a block. Examples of such assembled devices includes screws, nails, pins, rods, shafts of any cross-section (round or non-round), and any other device that is meant to pass into a cavity, the cavity created/formed in a block, that is to be prevented from backing out. As taught herein, embodiments described throughout the present disclosure may be used for a variety of orthopedic uses. For example, the present disclosure encompasses orthopedic plates (or blocks) are used with screws (or other suitable devices) to attach or fix the plates (or blocks) to bones, or to attach or fix the plates (or blocks) to other plates, which may then be implanted into the body.

According to an exemplary embodiment, the block may be defined broadly as any object with a cavity generally shaped or configured to receive a device to be inserted therein. The block, in an exemplary embodiment, may have at least one hole oriented generally orthogonal to the path of the device that is to be inserted into the cavity. In an exemplary embodiment, the device and/or block may be formed, manufactured or fabricated from: metal, plastic, ceramic and/or any other suitable material used in orthopedic applications. In an exemplary embodiment, the block may include two holes oriented generally orthogonal to the path of the device that is to be inserted into the cavity. In other exemplary embodiments, the block may include three holes oriented generally orthogonal to the path of the device that is to be inserted into the cavity.

According to an exemplary embodiment, one or more anti-backout features, which may be referred to as a locking wire or a locking feature throughout the present disclosure, may be placed into one or more of the previously described holes within the block. In an exemplary embodiment, the anti-backout feature, or locking feature, may be formed, manufactured or fabricated from metal, plastic, and/or any other suitable material capable of providing at least some elastic behavior. In one example, the at least one locking anti-backout feature may be positioned or inserted between two or more of the holes. By inserting the at least one locking feature into the one or more of the holes, a stop may be formed to prevent the device, as taught herein, from backing out of the cavity. In other words, the locking feature may be used to prevent the device from being ejected from the cavity of the block. To provide the most effective and/or greatest resistance to back out, and in an exemplary embodiment, it may be preferred that the at least one locking feature pass between two holes; thus, providing support of the locking feature at two locations.

The one or more locking features, as taught herein, may be formed as a cylindrical rod (or other suitable shape) that may be placed into the two holes as described. In such an exemplary embodiment, the two holes may be generally cylindrically adjacent to the cavity taught herein. Moreover, the two holes may be aligned with respect to each other such that the cylindrical axes of the two holes are co-linear in a region adjacent to the cavity taught herein. In other exemplary embodiments, there may be advantages to forming the one or more locking features as other shapes (e.g., pillars, tube-like shapes, conical, spherical, helical, or any other suitable shape useful during orthopedic applications). In such an embodiment using other shapes, a portion of both the locking feature and the hole must be matching in cross-section, and the size of both the hole and the locking feature generally does not change along this portion (e.g., a uniform cross-section is provided along the portion of both the locking feature and the hole).

The fundamental idea is to place a locking wire (defined as a locking feature herein), and position it in a non-linear hole to prevent an assembleable object (defined as a device in the disclosure) from backing out. The non-linear hole ensures that the locking wire won't fall out. The present disclosure may take advantage of 3D printing to create the cavities that previously would be difficult/impossible to manufacture.

The following embodiment provides an improved means to prevent back out of an assembled device from a cavity made or formed within a block. Such devices may include screws, nails, pins, rods or shafts of any cross-section (round and non-round) and any other device that is meant to pass into a cavity and must be prevented from backing out, where the cavity is created in a block. The block is defined broadly as any object with a cavity generally shaped to receive the device to be inserted. The block has at least one, and preferably two or more additional holes, oriented generally orthogonal to the path of the device as is it inserted in the cavity. One or more locking features are placed into one or more of the holes, or between two or more of the holes, forming a stop to prevent the device from backing out of the cavity. To provide the greatest resistance to back out, it is generally preferred that at least one locking feature pass between two holes, thus providing support of the locking feature at two locations.

In one preferred embodiment, the locking feature is a cylindrical rod that is placed into two holes. In this embodiment, the holes are generally cylindrical adjacent to the cavity, and aligned with each other such that the cylindrical axes of the two holes are co-linear in the region adjacent to the cavity. In other embodiments, there may be advantages to having the locking feature take other shapes. Important criteria in implementing the present disclosure is that there be a portion of both the locking feature and the hole that are generally matched in cross section, and that the size of both the hole and locking feature do not change along this portion.

The device and/or the block may be manufactured of metal, plastic, ceramic or other materials. The locking feature may be manufactured of metal or plastic or other materials that provide at least some elastic behavior.

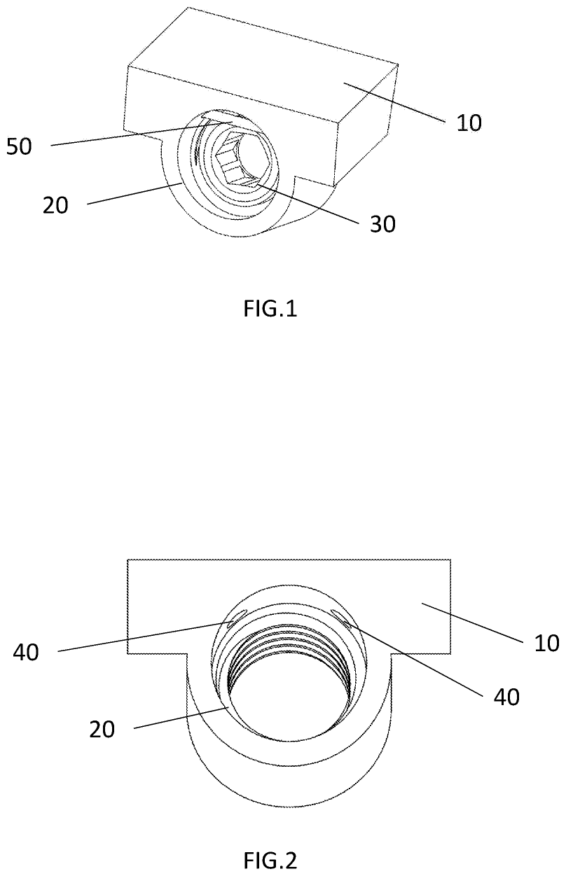

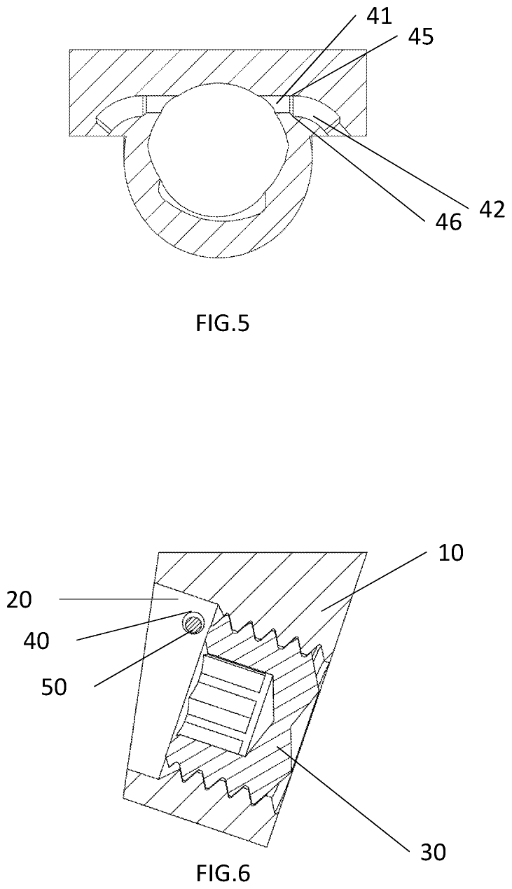

With reference now to FIGS. 1-6, an exemplary embodiment is depicted schematically. With initial reference to FIG. 1, a perspective view of an exemplary embodiment, depicted as a block 10, is shown. Block 10 may be formed with a cavity 20. In an exemplary embodiment, a device 30 (e.g., screw, nail, pin, rod, shaft having a round cross-section, shaft having a non-round cross-section, or any other suitable device as taught herein) may be seated or inserted into/within the cavity 20. An anti-backout feature or a locking feature 50 may be disposed or positioned within the cavity 20 and may be configured to prevent the device 30 from backing out of the cavity 20. It can be appreciated, consistent with aspects of the exemplary embodiments taught herein, that an additional object (not shown) may be provided to prevent the device from advancing through the cavity 20 in the direction opposite of the locking feature 50.

As shown in FIG. 2, which depicts the front view of the block 10, the cavity 20 is shown as a threaded hole. The threaded hole may be configured to receive a device 30 that is a screw (or other suitable device as taught herein) that may mate with the threads in the cavity 20. In an exemplary embodiment, two holes or openings 40 may be disposed along a surface of the block 10 with respect to the cavity 20, as shown. These holes 40 may be sized and/or dimensioned to receive the locking feature 50. In other exemplary embodiments, the block 10 may include more than two holes.

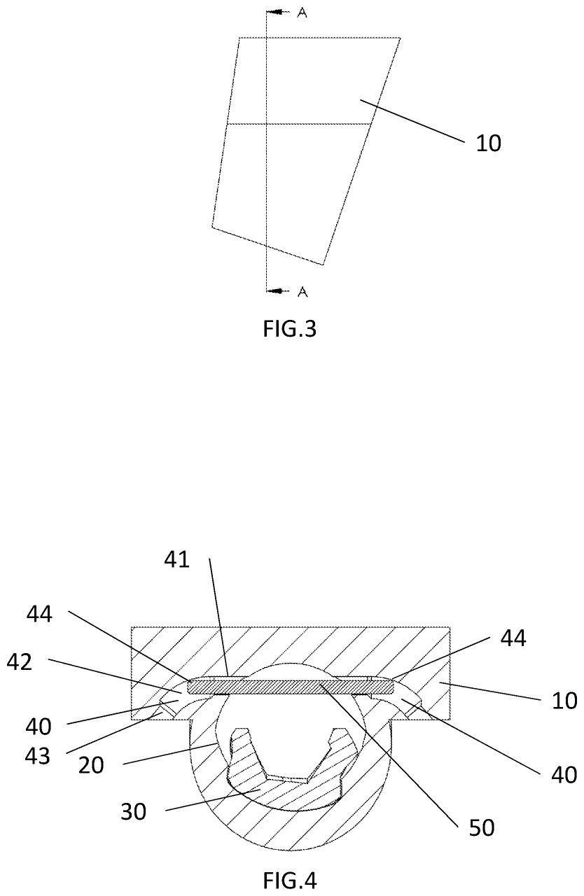

With reference now to FIG. 4, the embodiment of FIG. 1 is shown in a cross-sectional view as defined by line A-A in FIG. 3. In FIG. 4, all five elements are shown: the block 10, with the cavity 20, partially filled by the device 30, and two holes 40 that partially contain the locking feature 50. As shown, the locking feature 50 may be a straight rod that crosses or extends along the width of the cavity 20. In an exemplary embodiment, the rod is cylindrical and uniform in cross-section throughout the length of the rod. Also, as shown, first and second ends of the locking feature 50 or rod 50 may be seated within or received by the holes 40 on both sides of the cavity 20. In one embodiment, the holes 40 may include a size and/or cross-section that matches the size and/or cross-section of the associated end of the locking feature 50 that is to be received by the holes 40.

It can be appreciated that the shapes or cross-sections of the holes 40 are important, as are the complimentary matching shapes or cross-sections of the respective ends of the locking feature 50 (e.g., cylindrical rod). As previously taught herein, the shape and/or cross section of the holes 40 and the associated ends of the locking feature 50, that may be seated within the holes 40, may be identical to form a stop to prevent the device 30 from backing out of the cavity 20. In an exemplary embodiment, each hole 40 may be formed with at least two portions 41 and 42. In some embodiments, each hole 40 may be formed with additional portions, such as additional portion 43.

Hole first portion 41 may match the shape and/or size of a respective, associated end of the locking feature 50. In such an example, both the shape and/or size may be cylindrical. In some embodiments, however, the shape and/or size of the hole first portion may include a shape from a variety of different cross-sectional shapes (e.g., spherical, helical, non-rounded, etc.). In an exemplary embodiment, the hole second portion 42 may connect to the hole first portion 41. The hole second portion 42 may be partially misaligned relative to the hole first portion 41.

As shown in FIGS. 4-6, the hole second portion 42 may be curved in contrast to the straight hole first portion 41 and the straight locking feature 50. Alternatively, in some embodiments, the hole second portion 42 may be angled relative to the hole first portion 41. As best shown in FIG. 5, a transitional section 46 on a side of the block 10 away from a curved section 45 from the hole first portion 41 to the hole second portion 42 may be smooth in order to enable easy assembly of the locking feature 50 into the one or more associated holes 40 of block 10. The transitional section 46 on a side of the block 10 closer to the curved section 45 may be abrupt or smooth, as the locking feature 50 may not be expected to contact that side. The hole second portion 42 may be formed and/or dimensioned to be the same size as the hole first portion 41, or, in some embodiments, may be larger. A benefit of enlarging the hole second portion 42 relative to the hole first portion 41 may be to simplify the assembly, and require less bending of the locking feature 50. In some exemplary embodiments, additional hole portions 43 may be added as desired. In an exemplary embodiment, it may be desirable for the hole 40 to be a through hole, as opposed to a blind hole, which may be more difficult to clean. As shown, and according to an exemplary embodiment, the holes 40 may be planar. In some other exemplary embodiments, however, the holes 40 may be configured to be in different portions and/or may be positioned in a non-planar way to achieve the same function.

According to aspects of the present disclosure taught herein, and according to an exemplary embodiment, it may be desirable that the locking feature 50 extends beyond the curved section (e.g., a curvature junction) 45 in both holes 40. In such an example, the locking feature 50 may contact the side of the hole second portion 42 at a location 44, as shown in FIG. 4. This may result in a force applied to both ends of the locking feature 50, which may maintain its position in the holes 40 and may prevent the locking feature 50 from falling out of the block 10.

In an exemplary embodiment, it may be desirable to have the hole first portion 41 of the hole 40 adjacent to the cavity 20, and for the size of the hole first portion 41 of the hole 40 to be sized such that it may enable sliding of the locking feature 50. In such an example, this configuration may ensure the stiffest resistance to any force tending to dislodge the device 30 from the cavity 20, which may be known as backing out. However, there may be instances where a more gradual resistance to backout is preferred or desirable, in which case, a looser fit between the locking feature 50 and the hole first portion 41 of the hole 40 may be utilized.

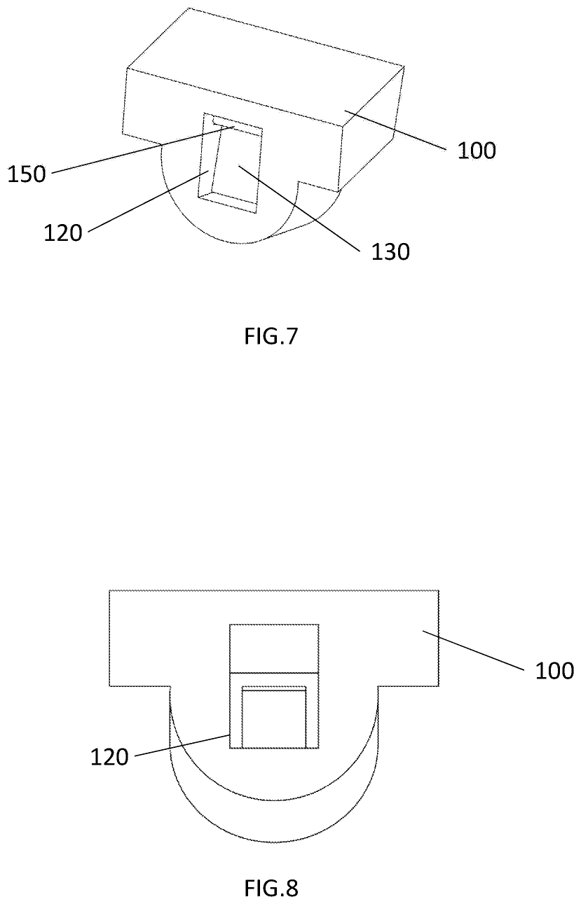

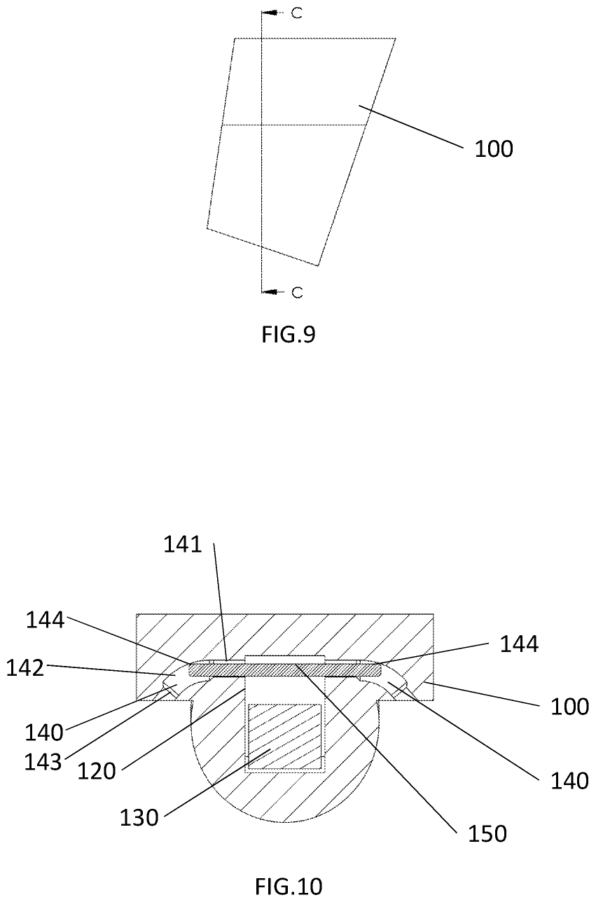

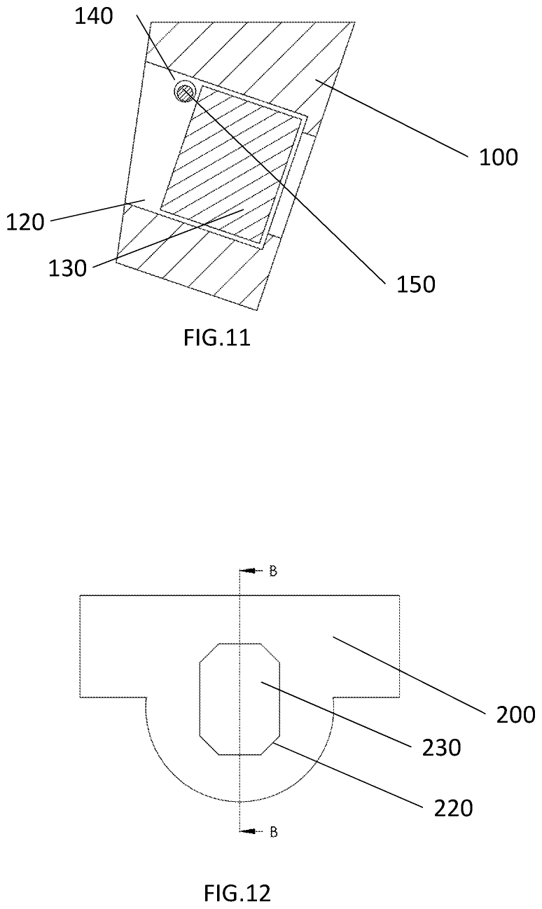

While the embodiment of FIGS. 1-6 show a generally circular cavity 20 with a generally circular device 30, other exemplary embodiments are possible. For example, FIGS. 7-11 depict an exemplary embodiment having non-circular features. As shown in FIG. 7, a block 100 may have a non-circular cavity 120. A device 130 may be seated or configured within the cavity 120. A device 130 may be seated deeper than a locking feature 150 within the block 100, as shown in FIG. 7. As shown in FIG. 10, two holes 140 may be disposed within the block 100. The holes 140 may extend generally orthogonal to a path that the device 130 follows as it is inserted into the cavity 120.

The holes 140 may include at least two portions—hole first portion 141 and hole first portion 142. Hole first Portion 141 may be formed adjacent to the cavity 120 while hole second portion 142 may be formed adjacent to hole first portion 141, but further removed from the cavity 120, as shown in FIG. 10. The hole first portion 141 may be generally sized and/or shaped to allow or permit a slideable motion between the hole first portion 141 and the locking feature 150. In some embodiments, the hole first portion 141 may be generally aligned with the locking feature 150.

In contrast, the hole second portion 142 may be sized and/or shaped and positioned so that the locking feature 150 contacts at least one side of the hole second portion 142, at positions 144, as shown in FIG. 10. A transition from hole first portion 141 to hole second portion 142 may be formed. In an exemplary embodiment, such a transition may be generally smooth on at least one side of the hole 140. On the second side of the hole 140, a transition from the hole first portion 141 to the hole second portion 142 may be abrupt or smooth. In an exemplary embodiment, the cross-sectional size and/or shape of the hole second portion 142 may be equal to or larger than that of the hole first portion 141. In some exemplary embodiments, in order to make the hole 140 a through hole, it may be desirable to include an additional portion 143 of the hole 140. Alternatively, in yet other exemplary embodiments, the hole second portion 142 may reach the outside of the block 100.

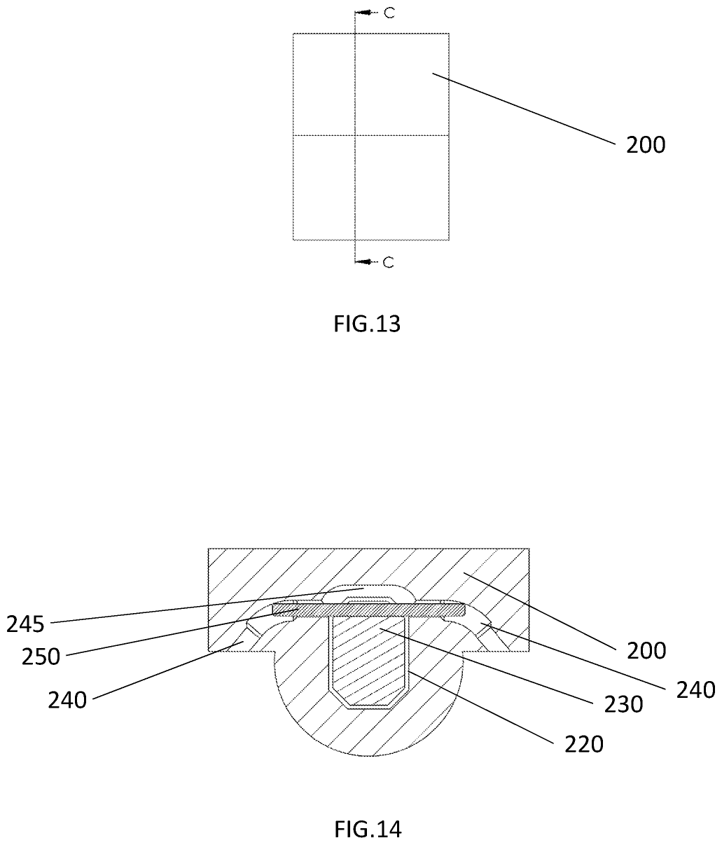

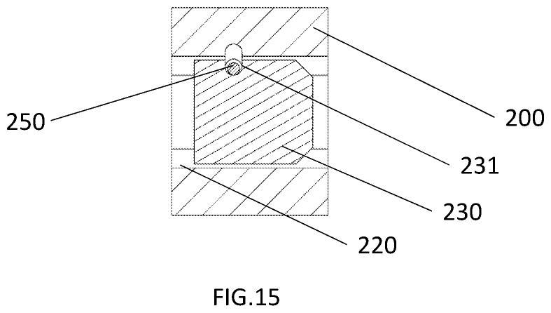

In yet some other exemplary embodiments, and according to aspects of the present disclosure taught herein, the position of a hole may be located in a cavity such that the axis of the hole intersects or nearly intersects a side of a device, for example, in an embodiment shown in FIGS. 12-15.

As shown in FIGS. 12-15, a block 200 may include a non-circular cavity 220. A device 230 may be seated or configured within the cavity 220, according to aspects of the disclosure taught herein. The device 230 may be positioned within the non-circular cavity 220 of the block 200 along with a locking feature 250. Two holes 240 may be formed in the block 200, and may be disposed generally orthogonal to a path that the device 230 follows as it is inserted into the non-circular cavity 220, but positioned to create interference with the device 230. In addition to the two holes 240, block 200 may include an additional opening 245. The opening 245 may be sized and/or shaped to receive the deformed locking feature 250 when assembling the device 230 into the block 200 with a pre-assembled locking feature 250. In addition, the device 230 includes a pocket 231 to receive the locking feature 250. When assembled, the locking feature 250 may engage the pocket 231 in order to prevent the device 230 from moving along the insertion path in either direction.

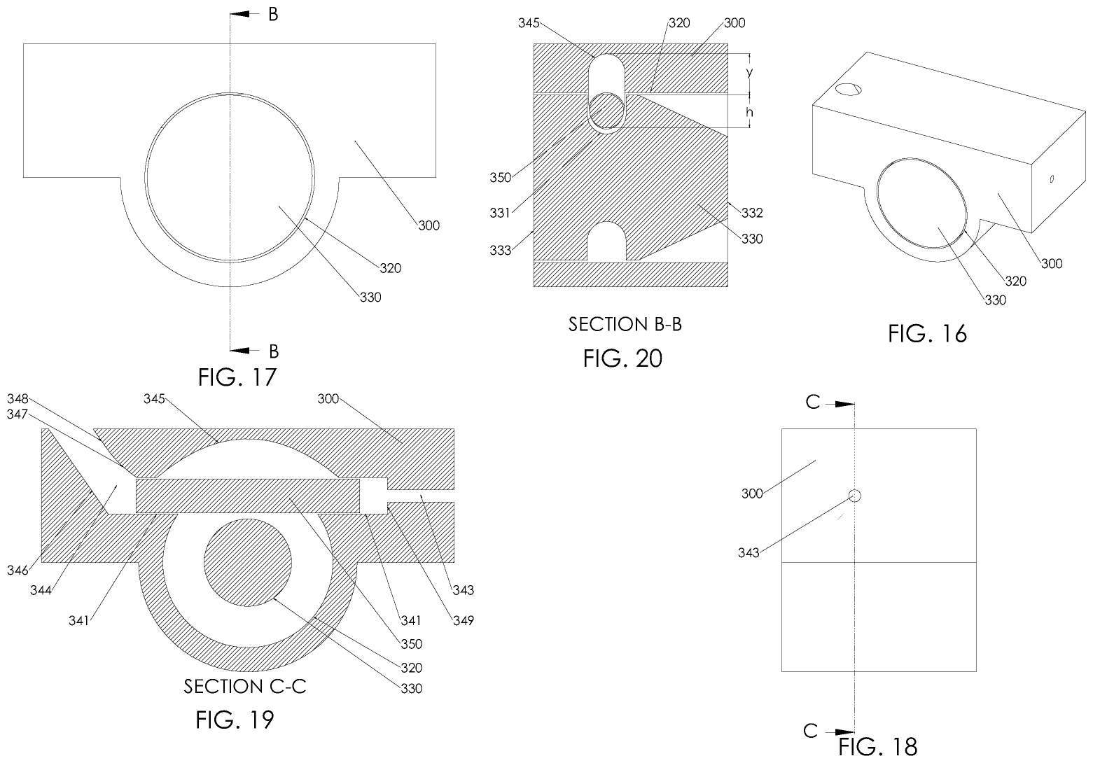

FIGS. 16-20 show an alternative embodiment. In this alternative embodiment, there are three components: a block 300, a device 330 and a locking feature 350. The locking feature 350 is pre-assembled in the block 300. Then, the device 330 may be assembled into the block 300 and by virtue of the locking feature, held in position.

The block 300 has a cavity 320 sized to receive the device 330. In addition, within the block, there is an opening 345 that is in communication with the cavity 320. The opening 345 is adjacent to hole first portions 341, which match the shape and/or size of a respective end of the locking feature 350 to allow or permit slideable motion between at least one hole first portion 341 and the locking feature 350. For example, in FIG. 19, there is no need for slideable motion between the locking feature 350 and the hole first portion 341 that terminates at second hole portion 343.

In this embodiment, two alternative hole second portions 343 and 344 are shown. Hole second portion 343 is sized to be smaller than the size of the locking feature 350, so as to prevent the locking feature from moving out of the block 300. Hole second portion 344 has a transition with hole first portion 341 that is abrupt on one side. The side 346 of hole second portion 344 meets hole first portion at an oblique angle. The benefit of this orientation is that it generally limits movement of the locking feature 350 if forces, for example rotation of the device 330, induce translation of the locking feature. The second side of hole second portion 344 is generally sized to permit the locking feature 350 to slide into the block 300. As shown, the second side is composed of a line 348 and an arc 347. The line 348 is generally parallel to the first side 346. The arc 349 provides clearance to permit the locking device 350 to be inserted through the hole second portion 344 and to align with hole first portions 341. Other geometries for the second side 347 may be considered to provide necessary clearance for insertion.

Alternatively, though less preferably, there could be no hole second portion 343 and the end of hole first portion 341 could terminate at a wall 349. This is less preferential as it results in a blind hole that can be more difficult to clean.

The locking feature 350 is a deformable member. The length of the locking feature 350 should fit between hole second portions 343 and 344. Further, it should be sufficiently long so that it sits within hole first portions 341, even when translated into contact with either a side of hole second portion 346, or the termination of hole first portion 349.

The device 330 has a leading end 332 and a trailing end 333. Between these ends, there is a pocket 331. The pocket 331 is sized to receive the locking feature 350. The pocket 331 is positioned to be aligned with the opening 345 and the hole first portions 341 in block 300 to position the device appropriately relative to the block 300, when the locking feature 350 rests within the pocket 331.

The opening 345 is sized to receive the locking feature 350 when it is deformed during insertion of the device 330 into the block 300. In cross-section as shown in FIG. 20, the shape of the opening generally conforms to the shape of the locking feature 350. Along the length of the locking feature 350, as shown in FIG. 19, the shape of the opening may be curved to generally mimic the shape of the deformed locking feature when displaced by the device 330 as it is inserted into the block 300. The displacement of a beam, such as the locking feature 350, is well known in mechanical engineering, where the vertical displacement (into the opening) is a function of the force applied to the beam, the beam material's elastic modulus, the beam's cross-sectional moment of inertia and the cube of the position along the length of the beam. Alternatively, the shape can be arcuate, or faceted or any combination thereof.

The opening size must be sufficient to allow the deformed locking feature to clear the device when the device is inserted into the cavity. Of course, for ease of manufacture or other reasons, the opening may be larger than the minimum size requirement. The opening height is defined as the distance from the edge of the device 330 to the aspect of the opening furthest from the device, as shown as dimension y in FIG. 20. The opening height may vary along the length of the locking feature. The minimum required opening height is equal to the depth of the locking feature below the outside surface of the device, as shown as dimension h in FIG. 20, when the parts are in their assembled position. It may vary along the length of the locking feature.

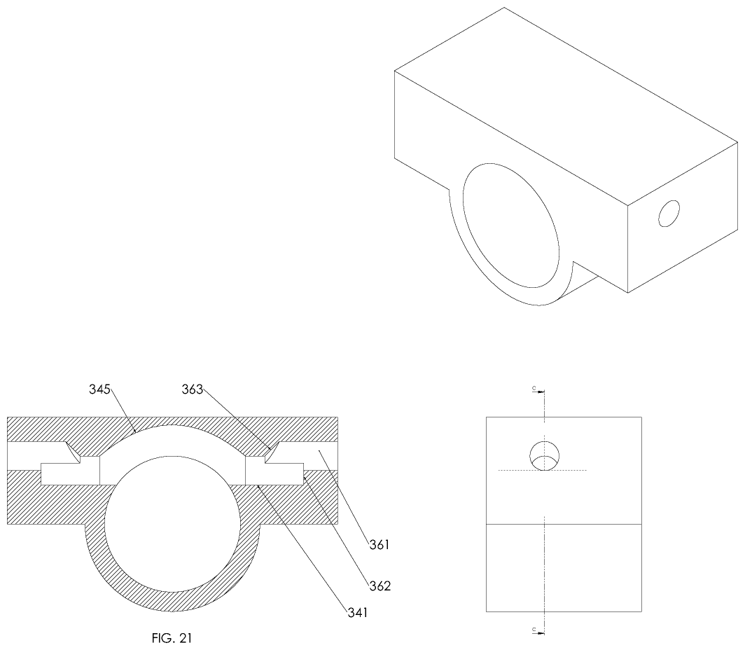

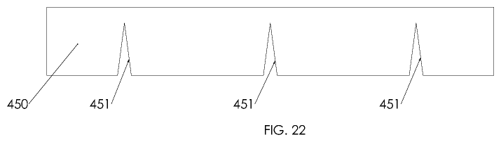

An alternate hole configuration is shown in FIG. 21. In this configuration, the first hole portion 341 and the second hole portion 361 are generally parallel. This results in a wall 362 generally perpendicular to the long axis of the locking feature 350 at the intersection of the first hole portion 341 and the second hole portion 361. The transition between the second hole portion 361 and the first hole portion 341, on the side opposite the wall 362 is a sloped surface 363. The slope could be curved or faceted. Also, note that along the length of the second hole portion there is an offset between the wall 362 and the sloped surface 363. This offset enables a deformable locking feature 350 to bend and enter its assembled position between the two first hole portions 341. Additionally, the wall 362 prevents displacement of the locking feature 350 in the direction of the second hole portion 361. As shown, the locking feature must be deformed to be inserted into the block, and in some embodiments, it must also be deformed to insert the device. To enhance deformation, alternative embodiments of the locking feature may be considered.

FIG. 22 shows a locking feature 450 where its elastic deformation has been enhanced by creating cutouts 451. These cutouts reduce the cross-section at these locations, enabling more elastic deformation at these sections. Depending on the requirements, the number of cutouts could vary. Additionally, the position of these cutouts along the length of the locking feature 450 can be adapted to provide appropriate deformation as needed.

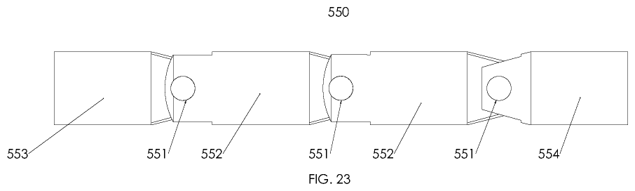

FIG. 23 shows a locking feature 550 composed of a series of links 552, 553 and 554, connected with pinned hinges 551. The hinges provide for rotation to enable the displacement of the locking feature during insertion of the locking feature 550 into the block 300, as well as during insertion of the device 330 into the block 300. The hinges 551 might also include a spring mechanism to generally align the links.

While principles of the present disclosure are described herein with reference to illustrative embodiments for particular applications, it should be understood that the disclosure is not limited thereto. Those having ordinary skill in the art and access to the teachings provided herein will recognize additional modifications, applications, embodiments, and substitution of equivalents all fall within the scope of the embodiments described herein. Accordingly, the invention is not to be considered as limited by the foregoing description.

What is claimed is:

1. An apparatus, comprising:
a block or plate formed with a cavity that is configured to receive a device, wherein the block or plate comprises at least two holes, and wherein each hole further comprises at least a first portion; and wherein said block or plate further comprises an opening adjacent to said cavity and to said holes; and
an elongate lockout feature comprising first and second ends, wherein the first and second ends define a cross-sectional profile, and wherein the first and second ends of the elongate lockout feature are seated within the respective at least two holes in the block or plate to prevent the device from backing out of the cavity of the block or plate.

2. The apparatus of claim 1, wherein a cross-sectional profile of the hole first portion has the same size, the same shape or the same size and shape as the cross-sectional profile of the first and second ends of the elongate lockout feature.

3. The apparatus of claim 1, wherein the hole first portion is connected to a hole second portion.

4. The apparatus of claim 3, wherein the hole second portion is partially misaligned relative to the hole first portion.

5. The apparatus of claim 3, wherein the hole first portion is straight, and wherein the hole second portion is curved relative to the straight hole first portion.

6. The apparatus of claim 1, wherein the block or plate is an orthopedic block or plate for attachment to bone.

7. The apparatus of claim 1, wherein the device is a medical device selected from the group consisting of a screw, a nail, a pin, a rod, a rounded shaft, or a non-rounded shaft.

8. The apparatus of claim 1, wherein the elongate lockout feature is a cylindrical rod.

9. The apparatus of claim 1, wherein the first hole portions of the at least two holes are co-linear.

10. The apparatus of claim 1, wherein the at least two holes are oriented generally orthogonal to a path of insertion of the device within the cavity.

11. A method for joining a block or plate to a device, comprising:
inserting a device into the block or plate formed with a cavity configured receive to device, wherein the block or plate is formed with at least two holes, and wherein each hole comprises a first portion and a second portion; and
securing the device within the block or plate by inserting an elongate lockout feature into the cavity of the block or plate, wherein the elongate lockout feature comprises first and second ends, wherein the first and second ends comprise a cross-sectional profile, and wherein the first and second ends of the elongate lockout feature are seated within the respective at least two holes in the block or plate to prevent the device from backing out of the cavity of the block or plate.

12. The apparatus of claim 11, wherein a cross-sectional profile of the hole first portion has the same size, the same shape or the same size and same shape as the cross-sectional profile of the first and second ends of the elongate lockout feature.

13. The apparatus of claim 11, wherein the hole first portion is connected to a hole second portion.

14. The apparatus of claim 13, wherein the hole second portion is partially misaligned relative to the hole first portion.

15. The apparatus of claim 13, wherein the hole first portion is straight, and wherein the hole second portion is curved relative to the straight hole first portion.

16. The apparatus of claim 11, wherein the block or plate is an orthopedic block or plate for attachment to bone.

17. The apparatus of claim 11, wherein the device is a medical device selected from the group consisting of a screw, a nail, a pin, a rod, a rounded shaft, or a non-rounded shaft.

18. The apparatus of claim 11, wherein the elongate lockout feature is a cylindrical rod.

19. The apparatus of claim 11, wherein the first hole portions of the at least two holes are co-linear.

20. The apparatus of claim 11, wherein the at least two holes are oriented generally orthogonal to a path of insertion of the device within the cavity.
